(19) United States
(12) Patent Application Publication
(10) Pub. No.: US 2011/0201859 A1
(43) Pub. Date: Aug. 18, 2011

(54) METHOD FOR PRODUCING CRYSTALLINE ZEOLITE-LIKE GALLO-ALUMINIUM SILICATES

(75) Inventors: Anna Omegna, Munchen (DE); Martin Claus, Munchen (DE)

(73) Assignee: SUD-CHEMIE AG, Munchen (DE)

(21) Appl. No.: 13/003,650

(22) PCT Filed: Jul. 10, 2009
(86) PCT No.: PCT/EP2009/005015

§ 371 Date: Mar. 31, 2011

(30) Foreign Application Priority Data
Jul. 11, 2008 (DE) 10 2008 032 699.2

(51) Int. Cl. C07C 2/00; C01B 39/02

(52) U.S. Cl. 585/407; 423/700

(57) ABSTRACT

A method for producing crystalline gallo-aluminium silicates, comprising the heating of a reaction mixture in a solvent, wherein the reaction mixture contains a silicon source, an aluminium source, a gallium source and a mineralization agent, wherein the reaction mixture comprises purely inorganic components and is free of nitrogen compounds. Further, aluminium silicates produced by the method according to the invention as well as the use thereof as catalyst.

### CROSS REFERENCE TO RELATED APPLICATIONS

This is a National Phase application of PCT application number PCT/EP2009/005015, filed Jul. 10, 2009, which claims priority benefit of German application number DE 10 2008 032 699.2, filed Jul. 11, 2008, the content of such applications being incorporated herein by reference.

### FIELD OF THE INVENTION

The present invention relates to a method for producing crystalline gallo-aluminium silicates, comprising the heating of a reaction mixture containing a silicon source, an aluminium source, a gallium source and a mineralization agent, in a solvent, wherein the reaction mixture comprises purely inorganic components. The invention further relates to the aluminium silicates produced by the method according to aspects of the invention as well as use thereof as catalyst.

### BACKGROUND OF THE INVENTION

By the term “zeolite” is meant within the framework of the present invention as defined by the International Mineralogical Association (D. S. Coombs et al., Can. Mineralogist, 35, 1997, 1571), a crystalline substance from the group of aluminium silicates with spatial network structure of the general formula

Mⁿ⁺[(AlO₂)ₓ(SiO₂)_{y]·(H}₂O)_{z}

which are composed of SiO₄/AlO₄ tetrahedra which are linked by common oxygen atoms to form a regular three-dimensional network. The Si/Al=y/x ratio is always ≧1 according to the so-called “Löwenstein rule” which prevents the occurrence of two adjacent negatively-charged AlO₄ tetrahedra. Although more exchange sites are available for metals at a low Si/Al ratio, the zeolite increasingly becomes more thermally unstable.

The zeolite structure contains cavities and channels which are characteristic of each zeolite. The zeolites are divided into different structures (see above) according to their topology. The zeolite framework contains open cavities in the form of channels and cages which are normally occupied by water molecules and extra-framework cations which can be replaced. An aluminium atom attracts an excess negative charge which is compensated for by these cations. The inside of the pore system represents the catalytically active surface. The more aluminium and the less silicon a zeolite contains, the denser is the negative charge in its lattice and the more polar its inner surface. The pore size and structure are determined, in addition to the parameters during production (use or type of templates, pH, pressure, temperature, presence of seed crystals), by the Si/Al ratio, which determines the greatest part of the catalytic character of a zeolite.

Because of the presence of 2- or 3-valent cations as tetrahedron centre in the zeolite framework the zeolite receives a negative charge in the form of so-called anion sites in the vicinity of which the corresponding cation positions are located. The negative charge is compensated for by incorporating cations into the pores of the zeolite material. Zeolites are differentiated mainly according to the geometry of the cavities which are formed by the rigid network of the SiO₄/AlO₄ tetrahedra. The entrances to the cavities are formed by 8, 10 or 12 “rings” (narrow-, average- and wide-pored zeolites). Specific zeolites show a uniform structural composition (e.g. ZSM-5 with MFI topology) with linear or zig-zag channels, while in others larger cavities attach themselves behind the pore openings, e.g. in the case of the Y and A zeolites with the FAU and LTA topologies.

In crystalline gallo-aluminium silicates, in addition to silicon and aluminium atoms, trivalent gallium atoms are also incorporated in the lattice. Tetrahedra comprised of oxygen atoms form a defined system of cavities with channels and pores, wherein the characteristic properties of the zeolite are defined by the size and number of these pores. If, for example, the M cations are replaced by protons after the synthesis of the zeolite, acid catalysts are obtained.

Catalysts based on crystalline gallo-aluminium silicates are used especially in the petrochemical industry for producing organic synthesis products. Due to their dehydrogenation and cyclization properties they are suited to converting low hydrocarbons from liquefied petroleum gas (LPG) such as e.g. alkanes, to aromatic hydrocarbons such as benzene, toluene or xylenes (so-called dehydrocyclodimerization).

In dehydrocyclodimerization processes crystalline gallo-aluminium silicate catalysts with a high SiO₂ content are principally used, in which x in the abovementioned general formula is greater than 12. These catalysts have a high degree of stability.

Many methods for producing crystalline, zeolite-like gallo(aluminium) silicates are known in the state of the art, wherein some methods describe incorporation of gallium into a completed zeolite lattice and other methods propose a direct synthesis of a gallo(aluminium) silicate via a hydrothermal crystallization of a synthesis gel.

### DESCRIPTION OF THE RELATED ART

U.S. Pat. No. 4,636,483 discloses for example a method for producing a catalyst based on a gallium-modified zeolite, wherein the gallium component is produced by impregnating calcined droplets which contain crystalline aluminium silicate and an aluminium oxide binding agent containing phosphorus compound, with an aqueous solution of a gallium metal salt.

EP 0 252 705 describes the introduction of gallium into catalytically active zeolites by treating a zeolite with an aqueous, gallium-containing medium under alkaline conditions or by means of ion exchange.

U.S. Pat. No. 4,861,933 discloses the production of a gallium-modified aluminium silicate zeolite by impregnation or ion exchange followed by calcining at least 700° C.

U.S. Pat. No. 6,593,503 discloses a method for producing a zeolite, wherein the zeolite is treated with acid in a first step, in order to reduce its aluminium content and, in a second step, is impregnated with a metal compound from the group of the compounds of nickel, palladium, molybdenum, gallium and platinum or combinations thereof, in order to obtain a metal-promoted zeolite.

EP 0 327 189 describes a method for producing a crystalline gallosilicate with MFI structure starting from a reaction mixture which contains a silicon source, a gallium source, alkali metals and an organic nitrogen-containing cation. The organic nitrogen-containing cation serves as template or structure-directing agent. However, a disadvantage when using templates of this type is that these must be removed by burning out following the synthesis of the zeolite or zeolite-like material. This therefore means an additional production step which is moreover associated with high energy costs. Additionally, some of the often toxic, nitrogen-containing templates remain in the mother solution, wherein the disposal of these mother solutions is likewise associated with increased costs. Additionally, the organic amines used are themselves very expensive, toxic and harmful to the environment.

The avoidance of organic compounds, in particular of organic amines as templates in zeolite synthesis would thus be advantageous.

As a solution to this, DE 41 20 847 A1 proposes a method for producing a zeolite-like pentasil gallosilicate, wherein no template is used. Instead, in this published document, the use of seed crystals during hydrothermal synthesis is described. The synthesis yield can thereby be increased without using a template. A disadvantage of this method is however, that in addition to the synthesis of the zeolite, the seed crystals have to be produced, which in turn takes place by the standard route via a synthesis gel and hydrothermal crystallization using an organic template. Such a method therefore makes no economic sense, has the abovementioned disadvantages and moreover substantially prolongs the reaction times.

U.S. Pat. No. 4,761,511 discloses a method for producing a pentasil-type gallo-aluminium silicate by hydrothermal crystallization of a synthesis gel, comprising a silicon source, an aluminium source, a gallium source, a mineralization agent, selected from oxides, hydroxides or salts of alkali or alkaline earth metals and an organic base. As organic base U.S. Pat. No. 4,761,511 discloses for example organic ammonium salts, amines or mono-, di- and trialkanolamines. As already stated above, these compounds are known as templates or structure-directing agents and bring with them the disadvantages already mentioned above.

### DESCRIPTION OF THE INVENTION

An object of the present invention was thus to provide a method for producing crystalline, zeolite-like gallo-aluminium silicates, wherein the synthesis is to take place without using organic templates or seed crystals.

The object is achieved by a method for producing crystalline gallo-aluminium silicates, comprising the heating of a reaction mixture in a solvent, wherein the reaction mixture contains a silicon source, an aluminium source, a gallium source and a mineralization agent, wherein the reaction mixture comprises purely inorganic components. In addition, the reaction mixture is free of nitrogen-containing compounds and free of seed crystals.

By the term “purely inorganic components” is meant that the reaction mixture is free of organic compounds, in particular free of organic and optionally inorganic amines and free of seed crystals. By free of nitrogen-containing compounds is meant in particular the absence of e.g. inorganic or organic amines, in particular also that the reaction mixture contains no NH₄⁺ions.

In particular it is also preferred that the reaction mixture is free of fluoride ions. By free of fluoride ions is meant within the meaning of this invention, that no fluoride ions, or only traces thereof, can be detected with the conventional analysis methods, but these have no influence on the reaction. Within the meaning of the invention the concentration of fluoride ions is only <500 ppm, preferably <250 ppm, most preferably <100 ppm.

Surprisingly, it was possible by the method according to aspects of the invention to obtain a crystalline gallo-aluminium silicate which can be used immediately. In other words, as the method according to aspects of the invention does not use a toxic and environmentally unacceptable organic template, it is therefore not necessary to burn out the template after the synthesis and the reaction product can be used immediately or further processed.

In the method according to aspects of the invention, silicon dioxide, sodium silicate, a silicon sol, silicic acid, colloidal silicic acid, precipitated silicic acid or pyrophoric silicic acid is preferably used as silicon source.

Aluminium oxide, sodium aluminate, aluminium hydroxide, or an aluminium salt preferably serves as aluminium source.

Gallium oxide, gallium hydroxide, a gallium salt or an alkali metal gallate is preferably used as gallium source.

An oxide, hydroxide or a salt of an alkali or alkaline earth metal is preferably used as mineralization agent. The mineralization agent Na₂O is particularly preferred.

The method is usually carried out by hydrothermal crystallization of the reaction mixture in a solvent at a temperature of more than 150° C., preferably of 155-250° C., particularly preferably at 170° C. over a period of more than 35 hours, preferably of more than 40 hours, particularly preferably in a period of 40 to 76 hours.

Water or a lower alkyl alcohol can be used as solvent. According to aspects of the invention lower alkyl means methyl-, ethyl, n-propyl or i-propyl.

The present synthesis is a one-step synthesis. This is both economically and technically advantageous, as the synthesis can be carried out more cost-effectively and more rapidly.

After completion of the synthesis the obtained zeolite is preferably filtered off, washed and dried at temperatures of approximately 100 to 130° C.

After drying, the obtained zeolite can be further processed directly, for example an ion exchange can also be carried out, i.e. in particular of the H⁺or Na⁺ions on the zeolite. The ion exchange, i.e. the replacement of additional metal ions such as Fe, Co, Ni, Mn etc. by H⁺or Na⁺, can be carried out via solid-state reactions or liquid-phase exchange known per se.

A subject of the invention is also the gallo-aluminium silicate produced by the method according to aspects of the invention. The gallo-aluminium silicate according to aspects of the invention wherein the majority of the gallium ions, i.e. more than 50%, are not situated in the zeolite lattice, but located in extra-lattice positions. This can be demonstrated e.g. by ⁷¹Ga MAS NMR. The gallium atoms or ions in the zeolite channels are preferably located at the ion-exchange positions in the immediate vicinity of the aluminium ions.

This only partial incorporation of the gallium into the framework, i.e. the presence of gallium at extra-lattice sites, proves advantageous when using the gallo-aluminium silicate according to aspects of the invention as catalyst, for example when aromatizing hydrocarbons, where a double functionality is necessary (Brønsted acidity and dehydrogenation function). Brønsted acidity is provided by the aluminium lattice atoms, whereas the dehydrogenation function of the gallium is provided at the extra-lattice sites. Due to its dehydrogenation and cyclization properties the gallo-aluminium silicate produced according to aspects of the invention is therefore particularly well suited to the conversion of lower hydrocarbons such as alkanes from liquefied petroleum gas (LPG) to aromatic hydrocarbons such as benzene, toluene or xylenes (so-called dehydrocyclodimerization).

The crystalline gallo-aluminium silicate preferably has a high SiO₂ content, particularly preferably, y is greater than 12 in the formula mentioned at the outset. The crystalline gallo-aluminum silicate produced according to aspects of the invention has a high degree of thermal stability.

### BRIEF DESCRIPTION OF THE DRAWING

The invention is explained in more detail below with reference to an embodiment example and a FIGURE which are not, however, to be considered limiting.

FIG. 1 shows ⁷¹Ga-NMR MAS spectra of a zeolite according to aspects of the invention and of a reference sample.

### DETAILED DESCRIPTION OF THE PREFERRED EMBODIMENTS

The ⁷¹Ga MAS NMR spectra were produced on an AVANCE 750 spectrometer in the 17.6 T field at a Larmor frequency of 228.6 MHz with MAS frequencies of 25 kHz.

The reference sample was a gallium-doped reference zeolite of MFI typology (ZSM-5) from: R. Klik et al., Zeolites, 19 (1997) 343-348.

Both samples were measured with a so-called HahnEcho (π/2, τ, π, τ), wherein the pulse interval τ corresponded to an MAS rotation period of 40 μs. The π/2 pulse had a length of 1 μs. Measurement took place with repeat periods of 1 s.

The reference zeolite had an Si/Ga ratio of 29. Gallium located in the MFI framework produces a signal approximately 20 ppm broad in the ⁷¹Ga MAS NMR spectrum at approximately 160 ppm.

The spectra of the zeolite according to aspects of the invention (example) as well as of the reference sample are represented at the same level. The true peak areas of both peaks for the same accumulation of approximately 50,000 are 100% for the sample H,Na[Ga]−ZSM−5 known from the abovementioned literature and 49% for Example 1.

An Si/Ga ratio of 59 thus results for Example 1.

Elemental analysis of Example 1 produced an Si/Ga ratio of 26. In other words, in addition to the gallium incorporated in the framework, gallium is present at extra-lattice sites, thus in a quantity of more than 50%.

Due to the cubic symmetry and thus the high quadrupole constant it is almost impossible to detect extra-framework gallium species in NMR (see reference above).

### Example 1

A preferred composition of a so-called synthesis gel (reaction mixture+solvent) according to the present invention is a mixture containing

16 equivalents of water,
1 equivalent of SiO₂,
0.027 equivalent of Al₂O₃,
0.006 equivalent of Ga₂O₃ and
0.1 equivalent of Na₂O.

The synthesis gel was converted to the finished product in one step, wherein the crystallization took place at 170° C. over 40 hours. The zeolite was then filtered off, washed and then dried at 120° C. and the yield determined. The obtained zeolite was a zeolite of MFI typology [H,Na[Ga]−ZSM5].

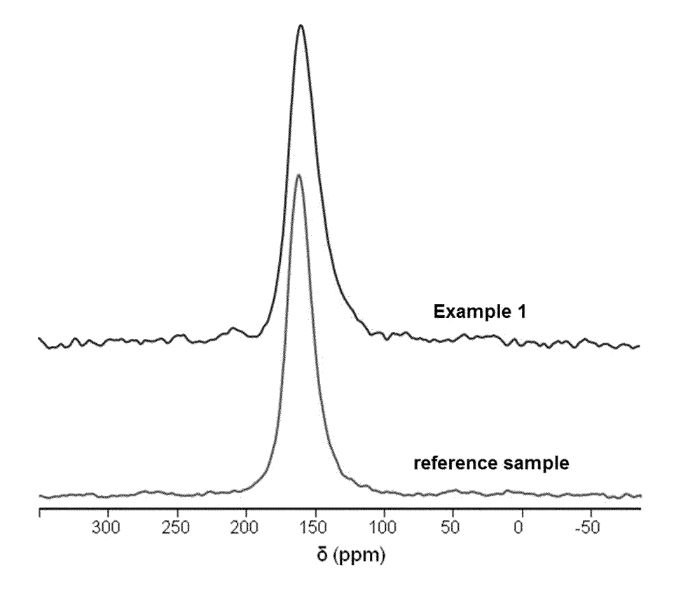

The product exhibits excellent filterability and thus also contributes to the high yield. The yield was 14% relative to the complete mixture. An elemental analysis of the mother solution showed that all the gallium was incorporated in the end-product with an Si/Ga ratio of 26. The ⁷¹Ga-NMR MAS is shown in FIG. 1.

1. A method for producing crystalline gallo-aluminium silicates, comprising the steps of heating a reaction mixture in a solvent, wherein the reaction mixture contains a silicon source, an aluminium source, a gallium source, and a mineralization agent, wherein the reaction mixture comprises purely inorganic components and is free of nitrogen-containing compounds and free of seed crystals.

2. The method according to claim 1, wherein the silicon source comprises one or more of silicon dioxide, sodium silicate, silicon sol, silicic acid, colloidal silicic acid, precipitated silicic acid or pyrophoric silicic acid is used as silicon source.

3. The method according to claim 1, wherein the aluminium source comprises one or more of aluminium oxide, sodium aluminate, aluminium hydroxide or an aluminium salt.

4. The method according to claim 1, wherein the gallium source comprises one or more of gallium oxide, gallium hydroxide, a gallium salt, or an alkali metal gallate.

5. The method according to claim 1, wherein the mineralization agent comprises one or more of an oxide, hydroxide, or salt of an alkali or alkaline earth metal.

6. The method according to claim 1, wherein the mineralization agent comprises Na₂O.

7. The method according to claim 1, wherein a hydrothermal crystallization is carried out at a temperature of more than 150° C. over a period of more than 35 hours.

8. The method according to claim 1, wherein the solvent comprises water or a lower alkyl alcohol.

9. Gallo-aluminium silicate, produced by the method according to claim 1.

10. Gallo-aluminium silicate, wherein more than 50% of the gallium ions are present at extra-lattice sites.

11. A method of organic synthesis comprising the gallo-aluminium silicate according to claim 9 as a catalyst.

12. The method of claim 2, wherein the aluminium source comprises one or more of sodium aluminate, aluminum hydroxide, or an aluminum salt.

13. The method of claim 12, wherein the gallium source comprises one or more of gallium oxide, gallium hydroxide, a gallium salt, or an alkali metal gallate.

14. The method of claim 13, wherein the mineralization agent comprises one or more of an oxide, hydroxide, or salt of an alkali or alkaline earth metal.

15. The method of claim 14, wherein the mineralization agent comprises Na₂O.
